(19) United States
(12) Patent Application Publication
(10) Pub. No.: US 2017/0080213 A1
(43) Pub. Date: Mar. 23, 2017

(54) COILED LEAD ASSEMBLY FOR NEUROSTIMULATION

(71) Applicant: PACESETTER, INC., Sylmar, CA (US)

(72) Inventors: Thomas Wright, Dallas, TX (US); Eric Eichelberger, Tualatin, OR (US); Paul Burros, West Linn, OR (US); Aaron Raines, Dalls, TX (US)

(21) Appl. No.: 14/859,031

(22) Filed: Sep. 18, 2015

(51) Int. Cl. A61N 1/05; A61N 1/36

(52) U.S. Cl. CPC A61N 1/0551; A61N 1/36071; A61N 1/36135

(57) ABSTRACT

An implantable neurostimulation lead comprises a lead body having a distal end and a proximal end, and a lumen extending along a longitudinal axis between the distal and proximal ends. Multiple electrodes are provided proximate to the distal end of the lead body. The electrodes are configured to at least one of deliver stimulating pulses and sense electrical activity. A multi-layer coil is wound about the lumen and extends at least partially along a length of the lead body. The multi-layer coil includes a first winding formed with multiple winding turn segments wound about the lumen in a first direction about the longitudinal axis. The multilayer coil includes a second winding formed with multiple winding turn segments wound about the first winding in an opposite second direction about the longitudinal axis.

### BACKGROUND OF THE INVENTION

Embodiments herein generally relate to lead assemblies for neural stimulation and more particularly to lead assemblies that support multiple sensing/therapy channels through a multi-layered coil structure.

Neurostimulation systems are devices that generate electrical pulses and deliver the pulses to nerve tissue to treat a variety of disorders. Spinal cord stimulation (SCS) is a common type of neurostimulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals, which are also electrically coupled to the wire conductors, that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator.

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

However, existing lead assemblies support a limited number of channels for sensing and delivery of therapy. Further, existing lead assemblies provide limited elongation capability when a patient moves.

### SUMMARY

An implantable lead comprises a lead body that is configured to be implanted in a patient. The lead body has a distal end and a proximal end, and a lumen extending along a longitudinal axis between the distal and proximal ends. A connector assembly is provided at the proximal end of the lead body. The connector assembly is configured to connect to an implantable medical device. Multiple electrodes are provided proximate to the distal end of the lead body. The electrodes are configured to deliver stimulating pulses and/or sense electrical activity. A multi-layer coil is wound about the lumen and extends at least partially along a length of the lead body. The multi-layer coil includes a first winding formed with multiple winding turn segments wound about the lumen in a first direction about the longitudinal axis. The multilayer coil includes a second winding formed with multiple winding turn segments wound about the first winding in an opposite second direction about the longitudinal axis.

Optionally, the second winding may represent an outer winding wound concentrically about an exterior surface of the first winding that represents an inner winding. Optionally, at least the first winding may include a plurality of wires that are electrically insulated from one another, the plurality of wires being joined to corresponding electrodes from the multiple electrodes. The first and second windings include a plurality of wires that are electrically insulated from one another and define a corresponding plurality of channels for at least one of sensing and delivering therapy. The first and second windings shift, along the longitudinal axis, between elongated and condensed states as a distance between the distal and proximal ends changes with patient movement.

In accordance with embodiments herein, the first and second windings have a first length when in a condensed state and a second length when in an elongated state, the second length being approximately 10% to 50% greater than the first length. The lead may comprise a low-friction inner layer provided about an interior surface of the first winding and a biocompatible outer layer provided about an exterior surface of the second winding. The inner and outer layers are formed of polymers to stretch and contract when the first and second windings elongate and condense with patient movement.

In accordance with embodiments herein, the lead may comprise a paddle structure provided on the distal end of the lead body, where multiple electrodes are arranged in an array on the paddle structure and individual wires within the first and second windings are electrically coupled to the corresponding electrodes.

In accordance with embodiments herein, an implantable system is provided that comprises an implantable medical device including pulse generating circuitry to generate electrical pulses as a neurostimulation therapy and a lead assembly. The lead assembly includes a lead body configured to be implanted in a patient, the lead body having a distal end and a proximal end, and a lumen extending along a longitudinal axis between the distal and proximal ends. A connector assembly is provided at the proximal end of the lead body. The connector assembly is configured to connect to the implantable medical device. Multiple electrodes are provided proximate to the distal end of the lead body, the electrodes configured to deliver stimulating pulses and/or sense electrical activity. A multi-layer coil is wound about the lumen and extending at least partially along a length of the lead body, the multi-layer coil including a first winding formed with multiple winding turn segments wound about the lumen in a first direction about the longitudinal axis, the multilayer coil including a second winding formed with multiple winding turn segments wound about the first winding in an opposite second direction about the longitudinal axis.

### BRIEF DESCRIPTION OF THE DRAWINGS

FIG. 1 depicts a stimulation system that generates electrical pulses for application to tissue of a patient according to one embodiment.

FIG. 2A depicts stimulation portions for inclusion at the distal end of lead in accordance with embodiments herein.

FIG. 2B depicts stimulation portions for inclusion at the distal end of lead in accordance with embodiments herein.

FIG. 2C depicts stimulation portions for inclusion at the distal end of lead in accordance with embodiments herein.

FIG. 3 illustrates a lead assembly formed in accordance with an embodiment.

FIG. 4A illustrates an end cross sectional view of the lead body formed in accordance with an embodiment.

FIG. 4B illustrates a side sectional view of a portion of the lead body taken along line A-A in FIG. 4A in accordance with embodiments herein.

FIG. 4C illustrates the multilayer coil in an at least partially elongated or extended state, in which at least a limited load is imposed upon the lead body in accordance with embodiments herein.

FIG. 4D illustrates a side view of a portion of the multi-later coil from the lead body to further illustrate relative motion in accordance with embodiments herein.

FIG. 5 illustrates a multi-layer coil having inner and outer windings in accordance with embodiments herein.

FIG. 6 illustrates a process for manufacturing a lead assembly in accordance with embodiments herein.

### DETAILED DESCRIPTION

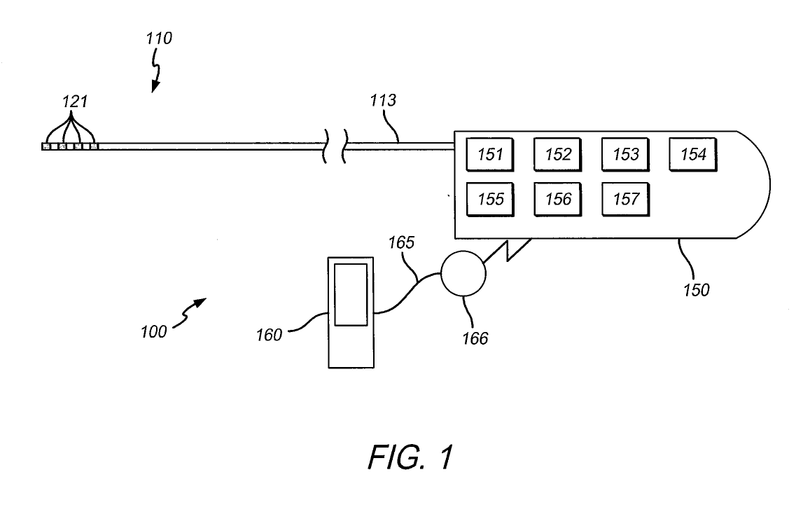

FIG. 1 depicts a stimulation system 100 that generates electrical pulses for application to tissue of a patient according to one embodiment. For example, system 100 may be adapted to stimulate spinal cord tissue, peripheral nerve tissue, deep brain tissue, cortical tissue, cardiac tissue, digestive tissue, pelvic floor tissue, or any other suitable tissue within a patient's body.

System 100 includes implantable medical device 150 that is adapted to generate electrical pulses for application to tissue of a patient. Implantable medical device (IMD) 150 typically comprises a metallic housing that encloses controller 151, pulse generating circuitry 152, charging coil 153, battery 154, far-field and/or near field communication circuitry 155, battery charging circuitry 156, switching circuitry 157, etc. of the device. Controller 151 typically includes a microcontroller or other suitable processor for controlling the various other components of the device. Software code is typically stored in memory of the IMD 150 for execution by the microcontroller or processor to control the various components of the device.

The IMD 150 is attached to a lead 110. For example, the lead 110 may connect with the “header” portion of the IMD 150 as is known in the art. Within the IMD 150, electrical pulses are generated by pulse generating circuitry 152 and are provided to switching circuitry 157. The switching circuit connects to outputs of the IMD 150. The terminals of one or more stimulation leads 110 are inserted within the IPG header for electrical connection with respective connectors. Thereby, the pulses originating from the IMD 150 are provided to stimulation lead 110. The pulses are then conducted through the conductors of lead 110 and applied to tissue of a patient via electrodes 121.

For implementation of the components within the IMD 150, a processor and associated charge control circuitry for an implantable pulse generator is described in U.S. Patent Publication No. 20060259098, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. patent Ser. No. 11/109,114, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within the IMD 150. Different pulses on different electrodes may be generated using a single set of pulse generating circuitry using consecutively generated pulses according to a “multi-stimset program” as is known in the art. Complex pulse parameters may be employed such as those described in U.S. Pat. No. 7,228,179, entitled “Method and apparatus for providing complex tissue stimulation patterns,” and International Patent Publication Number WO/2001/093953 A1, entitled “NEUROMODULATION THERAPY SYSTEM,” which are incorporated herein by reference. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 comprises a lead body 113 of insulative material about a plurality of wires/conductors within the material that extend from a proximal end of lead 110 to its distal end. The wires/conductors electrically couple a plurality of electrodes 121 to a plurality of terminals (not shown) of lead 110. The terminals are adapted to receive electrical pulses and the electrodes 121 are adapted to apply stimulation pulses to tissue of the patient. Also, sensing of physiological signals may occur through electrodes 121, the conductors, and the terminals. Additionally or alternatively, various sensors (not shown) may be located near the distal end of stimulation lead 110 and electrically coupled to terminals through conductors within the lead body 113. Stimulation lead 110 may include any suitable number of electrodes 121, terminals, and internal conductors.

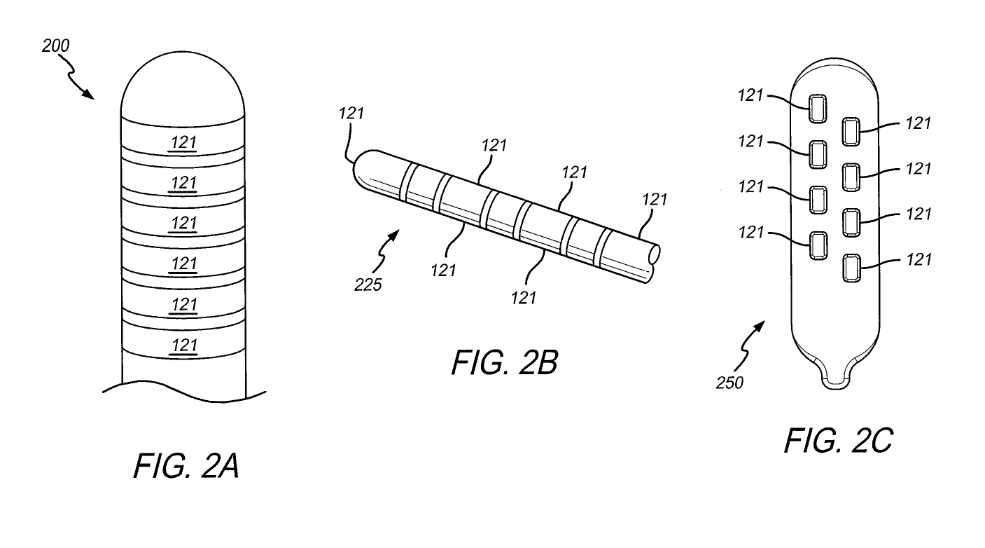

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. patent application Ser. No. 12/895,096, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Although not required for all embodiments, the lead bodies of lead(s) 110 may be fabricated to flex and elongate in response to patient movements upon implantation within the patient. By fabricating lead bodies according to some embodiments, a lead body or a portion thereof is capable of elastic elongation under relatively low stretching forces. Also, after removal of the stretching force, the lead body is capable of resuming its original length and profile. For example, the lead body may stretch 10% to 50% at forces of about 0.5 to 2.0 pounds of stretching force.

The ability to elongate at relatively low forces may present one or more advantages for implantation in a patient. For example, as a patient changes posture (e.g., “bends” the patient's back), the distance from the implanted pulse generator to the stimulation target location changes. The lead body may elongate in response to such changes in posture without damaging the conductors of the lead body or disconnecting from pulse generator. Also, deep brain stimulation implants, cortical stimulation implants, and occipital subcutaneous stimulation implants usually involve tunneling of the lead body through tissue of the patient's neck to a location below the clavicle. Movement of the patient's neck subjects a stimulation lead to significant flexing and twisting which may damage the conductors of the lead body. Due to the ability to elastically elongate responsive to movement of the patient's neck, certain lead bodies according to some embodiments are better adapted for such implants than some other known lead body designs. Fabrication techniques and material characteristics for “body compliant” leads are disclosed in greater detail in U.S. Provisional Patent Application Ser. No. 60/788,518, entitled “Lead Body Manufacturing,” filed Mar. 31, 2006, which is incorporated herein by reference.

Controller device 160 may be implemented to recharge battery 153 of the IMD 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery 153 by charging circuitry 154. Charging circuitry 154 may also communicate status messages to controller 160 during charging operations using pulse-loading or any other suitable technique. For example, controller 160 may communicate the coupling status, charging status, charge completion status, etc.

External controller device 160 is also a device that permits the operations of the IMD 150 to be controlled by user after the IMD 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate the IMD 150. The user interfaces may permit the user to move electrical stimulation along and/or across one or more stimulation leads using different electrode combinations, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME,” which is incorporated herein by reference. Also, controller device 160 may permit operation of IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

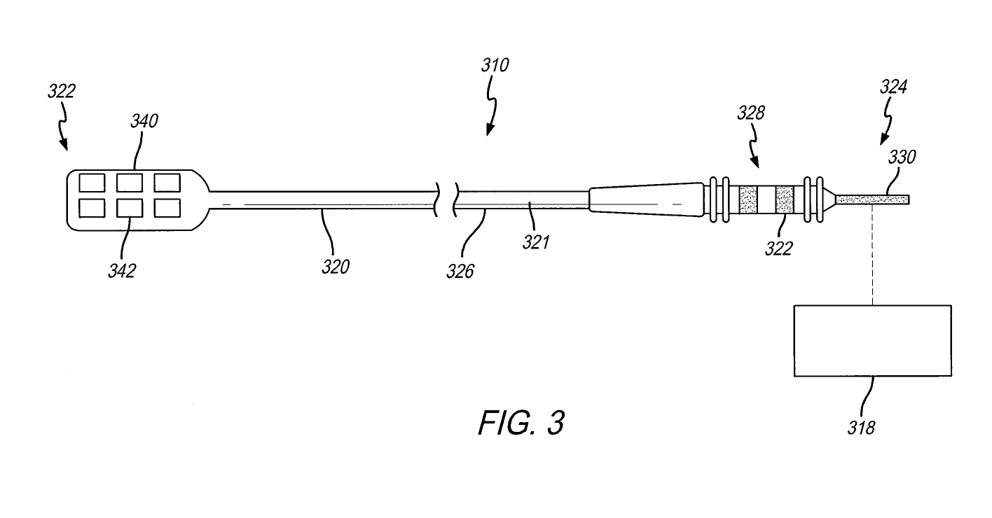

FIG. 3 illustrates a lead assembly 310 formed in accordance with embodiments herein. The lead assembly 310 includes an elongated lead body 320 which includes a distal end portion 322 and a proximal end portion 324. The lead body 320 has a length that extends along a longitudinal axis 321 between the distal and proximal end portions 322 and 324. The term longitudinal axis encompasses both linear and non-linear axes. The longitudinal axis of the lead body 320 extends along a curved path that changes as the lead body 320 is flexed, bent and otherwise manipulated. The lead body 320 includes an insulating sheath 326 of a suitable insulative, biocompatible, biostable material such as, for example, PEEK (i.e. Polyetheretherketones), silicone rubber or polyurethane, extending substantially the entire length of the lead body 320.

A connector assembly 328 is provided at the proximal end portion 324 of the lead body 312. The connector assembly 328 is configured to be inserted into a receiving orifice in the IMD 318. The connector assembly 328 includes first and second electrical terminals 330, 332 to be connected to respective electrical wires/conductors within the lead assembly 310. While first and second electrical terminals 330, 332 are illustrated, it is understood that multiple electrical terminals are provided at the connector assembly 328 for a corresponding number of individual channels supported by the lead assembly 310. For example, in an 8 channel lead, 8 separate electrical terminals are provided at the connector assembly 328. In a 16 channel or 32 channel lead, 16 or 32 electrical terminals may be provided at the connector assembly 328.

A paddle structure 340 is provided at the distal end portion 322 of the lead assembly 310. The paddle structure 340 includes an array of electrodes 342 at the distal end portion 322. The array of electrodes 342 may be arranged in a one-dimensional or two-dimensional array, with one or more electrodes associated with each individual channel supported by the lead assembly 310. For example, the paddle structure 340 may include a two-dimensional array of electrodes 342 having 2, 3, 5, 7 or more columns of electrodes with an equal, fewer or greater number of rows of electrodes. The electrodes 342 are electrically connected to corresponding electrical terminals. Optionally, the paddle structure 340 may also include a heat spreader provided thereon to convey thermal energy away from the paddle structure 340.

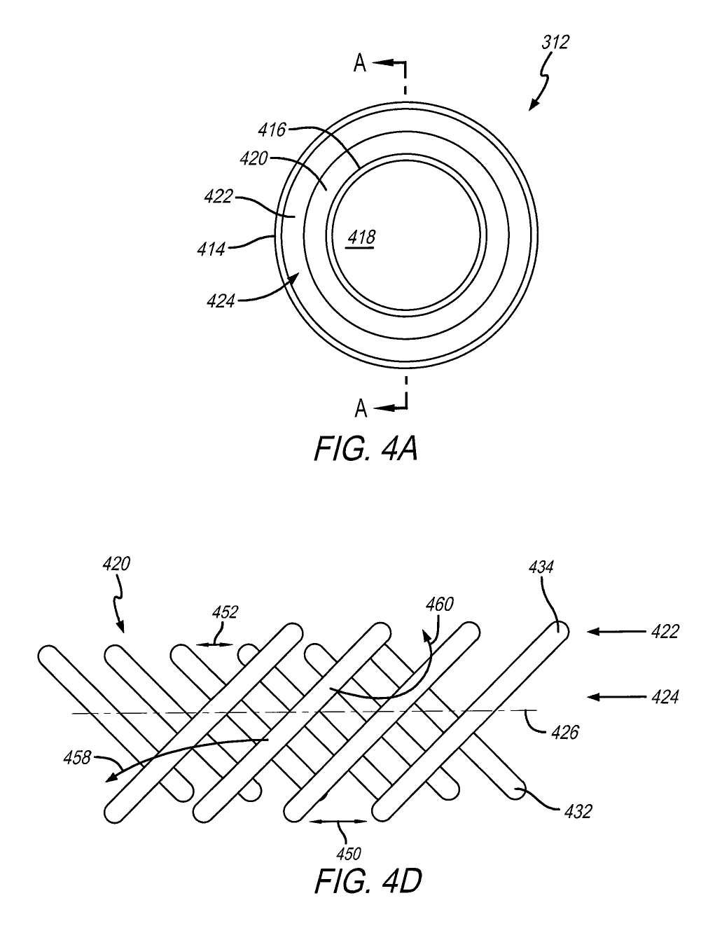

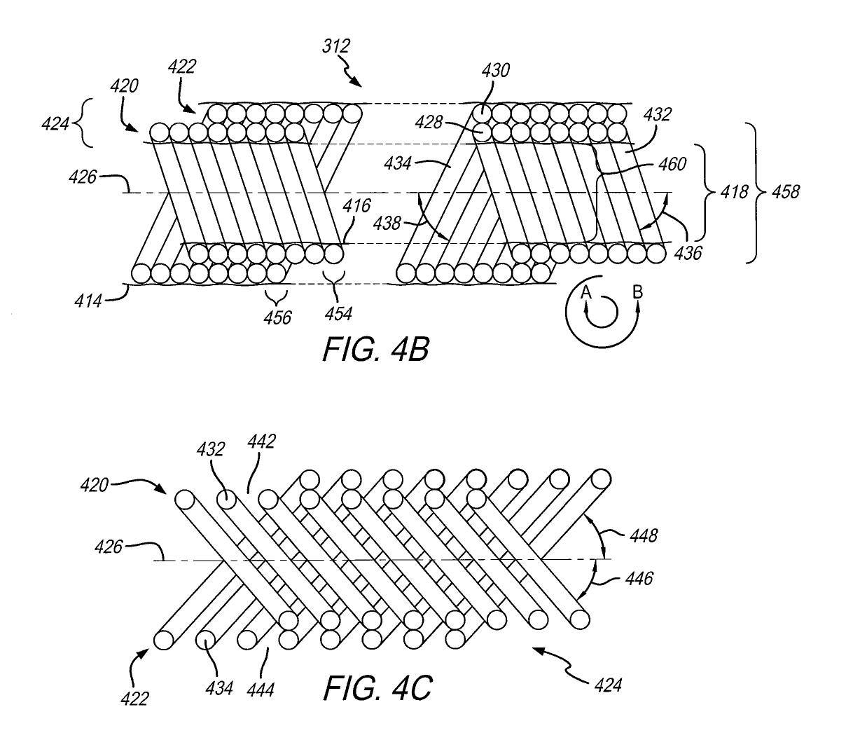

FIG. 4A illustrates an end cross sectional view of the lead body 312 (or lead body 113, FIG. 7) formed in accordance with an embodiment. The lead body 312 is symmetric and includes one or more inner lumen 418 configured to receive a guide wire, stylus or other device to facilitate implant of the lead body 312. The lumen 418 is defined by and surrounded by an inner sheath 416 and an outer sheath 414. The inner and outer sheaths 416 and 414 enclose inner (and sandwich there between) and outer windings 420 and 422. The inner and outer sheaths and windings 414-422 are arranged concentric with one another and extend along a longitudinal axis 426 of the lead body 312. The inner and outer sheaths 416 and 414 are formed of materials that stretch and contract when the lead body 312 is exposed to axial or radial forces such as during patient movement.

The inner sheath 416 may be formed with a tubular low-friction interior layer of a relatively high durometer, low friction, polymer-based material such as a Pellethane 55D material or other low-friction material configured to accommodate (and permit free movement of) a stylet or other guide device within the lumen 418. The interior layer is exposed to the lumen 418. The inner sheath 416 may also include a coating of a biocompatible layer of an elastomeric polymer, such as Carbosil 80A, where the biocompatible layer is coated over an exterior surface of the low-friction interior layer formed by the Pellethane 55D. Optionally, other materials may be used in place of the Pellethene 55D material or the Carbosil 80A material.

The outer sheath 414 is a tubular layer formed of a biocompatible material such as by re-flowing a fluorinated ethylene propylene (FEP) heat shrinking tubing over the outer winding 422. Optionally, the number and configuration of lumen may vary depending upon the type of lead. The multi-layer coil may be used in a variety of lead types with a variety of lumen positions and shapes.

The inner and outer windings 420 and 422 collectively define a multi-layer coil 424 formed in accordance with embodiments herein. The multi-layer coil 424 extends at least partially along a length of the lead body 312. For example, the coil 424 may be utilized in a limited region only proximate to the distal end of the lead body 312, or in a region proximate to the distal end and intermediate portion of the lead body 312. Optionally, the coil 424 may extend along the entire length of the lead body 312. The inner winding 420 is located concentrically within the outer winding 422. Each of the windings 422 and 420 are structurally continuous, conductive wires (also referred to as filars) that electrically define channels that are utilized to convey sensing signals and/or deliver therapy between the electrode array and the IMD.

FIG. 4B illustrates a side sectional view of a portion of the lead body 312 taken along line A-A in FIG. 4A. FIG. 4B illustrates the inner sheath 416 extending along the longitudinal axis 426 of the lead body 312 and about the lumen 418. The multilayer coil 424 is provided about the inner sheath 416 and comprises at least the inner and outer windings 420 and 422. As illustrated in FIG. 4B, the outer winding 422 is wound concentrically about an exterior surface of the inner winding 420. Optionally, more than two windings may be provided concentrically over one another, such as by adding third or fourth layers of windings concentrically arranged about the outer winding 422. The outer sheath 414 is re-flowed over and joined through heat shrinking to the outer winding 422.

The first or inner winding 420 includes a plurality of filars/wires 428 that are electrically insulated from one another. The second or outer winding 422 also include a plurality of filars/wires 430 that are electrically insulated from one another. Each wire 428, 430 comprises one or more conductive strands coated with an insulating layer such as an Ethylene tetrafluoroethylene (ETFE) layer which has a relatively high melting temperature and high resistance properties. Optionally, the ETFE insulation layer may be further coated with a biocompatible layer, such as Carbosil 80A. Each of the wires 428 and 430 may comprise an individual conductive strand, or a group of conductive strands bound together within the insulation layer.

The wires 428 have a common wire outer diameter 454. The wires 430 have a common wire outer diameter 456. The wire outer diameters 454 and 456 are defined by the conductive strands, surrounded by an insulation layer and further surrounded by a biocompatible layer. The relation between the wire outer diameters 454, 456 may be varied by adjusting the thickness of the insulation layer and/or biocompatible layer. In accordance with embodiments herein, the wire outer diameter 454 of the inner wires 428 forming the inner winding 420 is less than the wire outer diameter 456 of the outer wires 430 forming the outer winding 422 by utilizing a thinner biocompatible layer for the inner wires 428, as compared to the thickness of the biocompatible layer provided on the outer wires 430. The wire outer diameter 454 is defined to be less than the wire outer diameter 456 to maintain a desired relation between the pitch angles at which the inner windings 420 and outer windings 422 are oriented relative to the longitudinal axis 426. For example, it may be desired to maintain a common pitch angle for the inner and outer windings 420, 422, and thus to do so, the wire outer diameter 456 is increased, relative to the wire outer diameter 454 by increasing the thickness of the biocompatible layer (and/or insulation layer). By way of example only, the wire outer diameter 454 may be 0.006 inches, and the wire outer diameter 456 may be 0.008 inches. The wire outer diameter 456 is made larger than the wire outer diameter 454 by increasing the amount of insulation surrounding the conductive strands, such as when it is desirable to maintain a common diameter for the conductive strands within the inner and outer windings 420, 422. By increasing or decreasing the thickness of the insulation layer about the conductive strands in the outer wires 430, the wire outer diameter 456 may be managed. The outer winding 422 has a larger core diameter 458 (as measured orthogonal to the axis 424), as compared to the core diameter 460 of the inner winding 420. To facilitate manufacturing, the wire outer diameter 456 is enlarged relative to the wire diameter 454 in part based on the difference in the core diameters 458 and 460.

The wires 428 and 430 are electrically joined, at the distal end, to corresponding electrodes (e.g. provided on leads in FIGS. 2A-2C or the paddle structure 340 in FIG. 3), and are electrically joining, at the proximal ends, to electrical terminals (e.g. the connector assembly 328 in FIG. 3). In accordance with some embodiments, each wire 428, 430 defines a separate channel over which one or both of sensed signals and therapy stimulations are conveyed. Additionally or alternatively, combinations of the wires 428 and/or combinations of the wires 430 may be joined within a single channel. By way of example only, when a 16 channel lead is desired, 8 individual wires 428 may be provided within the inner winding 420 to define 8 channels, and 8 individual wires 430 may be provided within the outer winding 422 to define an additional 8 channels. It is recognized that the number of individual wires 420, 430 will vary based upon the number of channels desired.

The inner winding 420 is formed from multiple winding turn segments 432 wound in a first direction (e.g. left hand wrapped) about a mandrel extending along the longitudinal axis 426. The outer winding 422 is formed from multiple winding turn segments 434 that are wound in an opposite second direction (e.g. right hand wrapped) about the mandrel and the longitudinal axis 426. By way of example, the winding turn segments 432 may be wound in a clockwise direction as denoted by arrow A about the longitudinal axis 426, while the winding turn segments 434 may be wound in a counterclockwise direction as denoted by arrow B about the longitudinal axis 426.

Optionally, as the wires 428 are wound about a mandrel, “backtwist” compensation may be introduced into the wires 428 to facilitate maintaining the wire 428 upon the mandrel (e.g., to neutralize a wire's tendency to unwind). Backtwist compensation represents a slight rotation introduced along the length of the wire 428 while being wound about the mandrel. Similarly, backtwist compensation may be introduced into the wires 430 as wound about the exterior surface of the inner winding 422 to neutralize the wire's tendency to unwind. The directions of the backtwist compensation may be opposite one another, based upon the direction in which the wires 428 and 430 are wound about the longitudinal axis 426.

In accordance with embodiments herein, the multilayer coil 424 affords a great capacity for elongation without exposure to lead fracture and in response to relatively low forces. By providing a greater level of elongation, the lead body 312 reduces a degree to which loading is introduced at the anchoring sites during patient movement. For example, when a patient bends, twist or otherwise moves, the lead body 312 exhibits relatively limited or no loading at the distal or proximal ends of the lead, thereby avoiding potential movement at a paddle structure electrode, or any other type of electrode assembly at the distal end. It is desirable to avoid movement of the electrode assembly as such movement may affect the pattern of stimulation. For example, the lead avoids placing tension on a paddle structure or other electrode configuration, such as in an epidural space, in a deep brain implant location, and elsewhere. The lead body 312 also avoids loading at the proximal end where the IMD is position. It is desirable to avoid movement at the IMD as such movement may be felt by the patient and be judged uncomfortable (e.g. when the IMD is located in the abdomen or elsewhere subcutaneously). By way of example, configurations described herein may exhibit 10% to 50% elongation of the lead body relative to an overall length of the lead body when in a fully contracted or condensed state (e.g. as measured between the proximal and distal ends of the lead). While the overall lead body may exhibit 10% to 50% elongation relative to a fully contracted state, within the lead body, the inner and outer winding 420 and 422 may extend up to 50% beyond a fully contracted state.

The inner and outer windings 420 and 422 shift, along the longitudinal axis 426, between elongated and condensed states as a distance between the distal and proximal ends (e.g. 322 and 324 in FIG. 3) change with patient movement after implantation of the lead. The inner and outer windings 420 and 422 also shift as the lead body 312 is bent laterally in response to patient movement and/or during implant. The transition of the multilayer coil 424 between elongated and condensed states described hereafter further in connection with FIGS. 4B and 4C.

FIG. 4B illustrates the multilayer coil 424 when in a condensed or contracted state, in which no or a relatively limited load is imposed upon the lead body 312. As shown in FIG. 4B, when in the condensed or contracted state, the individual turn segments 432 of the inner winding 420 are positioned substantially adjacent one another and abut against one another such that successive turn segments 432 form a substantially continuous and uninterrupted inner turn structure. Similarly, the individual turn segments 434 of the outer winding 422 are positioned substantially adjacent one another and abut against one another such that successive turn segments 434 form a substantially continuous and uninterrupted outer turn structure. The turn segments 432 are coiled “line to line” such that no gaps exist between the wires 428. Similarly, the tune segments 434 are coiled “line to line” such that no gaps exist between the wires 430.

The turn segments 432 of the inner winding 420 are wound about a mandrel (and the lumen 418) at a select pitch angle (also referred to as a condensed pitch angle) relative to the longitudinal axis 426. The pitch angle represents the rise over run. For example, the turn segments 432 are oriented at an acute pitch angle 436 of between 30 and 50° relative to the longitudinal axis 426. As a further example, the turn segments 432 may be oriented at an acute pitch angle 436 of approximately 30°. The turn segments 434 of the outer winding 422 are wound about inner windings 420 at a select pitch angle relative to the longitudinal axis 426. For example, the turn segments 434 are oriented at an acute pitch angle 438 of between 30 and 50° relative to the longitudinal axis 426. As a further example, the turn segments 434 may be oriented at an acute pitch angle 436 of approximately 30°. The first and second pitch angles 436 and 438 may be the same (e.g., common) or may be different, depending upon various factors, such as the difficulty of manufacture, the size of the wires 428, 430, the number of winding layers, the level of elasticity desired in the lead and the like. The pitch angles 436, 438 may be adjusted, based on the degree to which the pitch angle renders the inner and outer windings 420, 422 difficult to manufacture or no longer stretches without applying undue force to the electrodes and/or IMD.

FIG. 4C illustrates the multilayer coil 424 in an at least partially elongated or extended state, in which at least a limited load (e.g., a stretching force and/or a bending force) is imposed upon the lead body 312. When a load is imposed upon the lead body 312, the windings 420 and 422 expand along the longitudinal axis 426 in a scissoring action. When in the elongated or extended state, the individual turn segments 432 of the inner winding 420 are positioned in a spaced apart manner with gaps 442 there between to form a spread or a distributed turn structure. Similarly, the individual turn segments 434 of the outer winding 422 are positioned in a spaced apart manner with gaps 444 there between to form a spread or a distributed turn structure.

When in the elongated or extended state, the turn segments 432 are oriented at a pitch angle 446, relative to the longitudinal axis 426, that is less acute than the pitch angle 436 (corresponding to the condensed state in FIG. 4B). The turn segments 434 of the outer winding 422 are also oriented at a pitch angle 448, relative to the longitudinal axis 426, that is less than the pitch angle 438. For example, the pitch angles 446, 448 may be between 60 and 40°. As a further example, when the pitch angles 436, 438 is 40°, the pitch angles 446, 448 may be 50°.

It is recognized that the turn segments 432, 434 expand in various manners depending upon the movement of the lead body. For example, portions of the turn segments 432, 434 may stretch linearly along the longitudinal axis 426, while other portions of the turn segments 432, 434 may bend in an arcuate shape or remain condensed.

FIG. 4D illustrates a side view of a portion of the multi-later coil 424 from the lead body to further illustrate relative motion in accordance with embodiments herein. FIG. 4D illustrates the turn segments 434 of the outer winding 422 as overlapping and encompassing the turn segments 432 of the inner winding 420. As the turn segments 434 and 432 separate during expansion, overlapping turn segments 432 and 434 move in a scissoring action relative to one another along the longitudinal axis 426. In the scissoring action, the turn segments 432 formed one portion of the scissor, while the turn segments 434 form the opposed portion of the scissor. Adjacent turn segments 434 stretch in a helical manner in an axial direction 450, while turn segments 432 stretch in a helical manner in an axial direction 452. The axial directions 450 and 452 may be parallel to the longitudinal axis 426, depending upon the type of movement by the patient. The turn segments 432 and 434 may stretch and compress in a helical manner along various non-lineal arcuate paths, such as denoted by arrows 458 and 460 depending upon the patient movement.

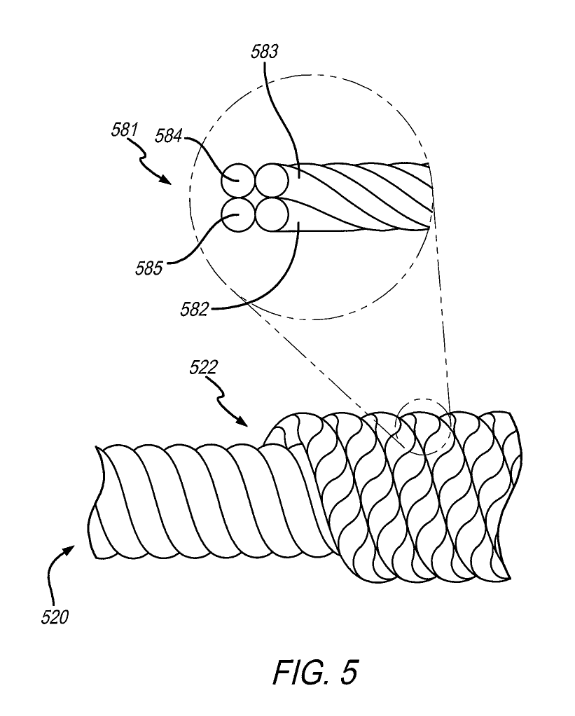

FIG. 5 illustrates a multi-layer coil having inner and outer windings 520 and 522. One or both of the inner and outer windings 520 and 522 are formed from bundles 581 of filars or wires 582-584. The bundles 581 are wound into a spiral shape to form the inner or outer windings 520, 522. In FIG. 5, the bundle 581 of individual filars/wires 582-584 is twisted in a helix. The bundle 581 may then be wound about the mandrel to form the inner winding 520 and/or outer winding 522. The wires 582-584 may each correspond to a separate channel. Alternatively, the wires 582-584 may be joined to be electrically common to form a single channel. By way of example, if a bundle 581 were used for the inner and outer windings 420 and 422, with each wire 582-584 remaining electrically separate from one another, a lead may be provided with 64 individual channels, by utilizing 8 separate turn segments 432 in the inner winding 420, and 8 separate turn segments 434 in the outer winding 422, where each winding comprises 4 individual wires 582-584.

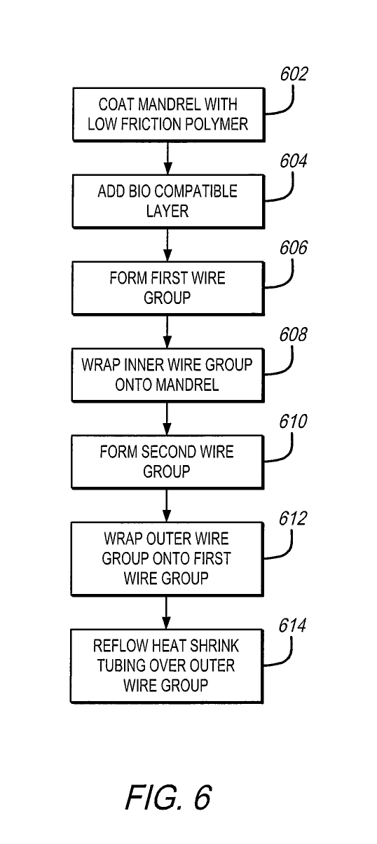

FIG. 6 illustrates a process for manufacturing a lead assembly in accordance with embodiments herein. Beginning at 602, a mandrel is provided having an outer diameter corresponding to a diameter of the lumen to be formed within the lead assembly. At 602, the mandrel is coated with a high durometer polymer that exhibits low friction, such as a Pellethene 55D material. At 604, an outer surface of the low friction polymer is coated with a biocompatible elastomeric polymer, such as a Carbosil 80A material. At 606, the individual wires are formed with conductive strands coated with an insulation layer that is coated with a biocompatible real. At 606, the wires are formed into a first group (e.g. a group of 8). Optionally, each of the wires in the group may be coated with a different color of polymer or otherwise individually marked to afford wire identification. At 608, the first wire group is wrapped about the mandrel to form the inner winding 420 (FIGS. 4A-4D). While the wires within the first wire group are being wrapped, each individual wire is slightly twisted along the corresponding wire axis to neutralize the wire's tendency to unwind. As the first wire group is wrapped, each of the individual wires are maintained adjacent one another and abutting against one another such that no gaps exist there between. Heat shrink tubing is utilized to hold the first wire group in a wrapped state upon the mandrel.

At 610, the second wire group is formed such as by providing an insulation coating over the conductive stands and providing a biocompatible coating layer over the insulation coating. The wires in the second group may also be coated with different colors of polymers or otherwise individually marked to afford wire identification. At 612, the second wire group is wrapped about the mandrel to form the outer winding 422 (FIGS. 4A-4D). While the wires within the second wire group are being wrapped, each individual wires slightly twisted along the corresponding wire axis to neutralize the wire's tendency to unwind. As the second wire group is wrapped, each of the individual wires are maintained adjacent one another and abutting against one another such that no gaps exist there between. The second wire group is wrapped in a direction opposite to the wrapping direction of the first wire group. For example, if the first wire group is wrapped in a clockwise direction, the second wire group is wrapped in a counterclockwise direction, or vice versa. At 614 an FEP heat shrink tubing is reflowed over the outer wire group to create a homogeneous lead body with no voids or bubbles between wires.

The foregoing processes and structures provide a lead assembly that exhibits a greater level of elongation capability relative to pre-existing lead assemblies in which all conductors are wound in a common winding group. By increasing the level of elongation, embodiments herein reduce the level of loading placed upon anchoring sites (e.g. the electrode array and the IMD) during patient movement. In addition, embodiments herein achieve a smaller diameter, thereby providing a lower profile lead assembly, as compared to other solutions that may result if all conductors are wound in a common winding group.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

1. An implantable lead, comprising:
a lead body configured to be implanted in a patient, the lead body having a distal end and a proximal end, and a low friction inner sheath forming a lumen extending along a longitudinal axis between the distal and proximal ends;
a connector assembly provided at the proximal end of the lead body, the connector assembly configured to connect to an implantable medical device;
multiple electrodes provided proximate to the distal end of the lead body, the electrodes configured to at least one of deliver stimulating pulses and sense electrical activity; and
a multi-layer coil wound about the lumen and extending at least partially along a length of the lead body, the multi-layer coil including a first coil layer having a first winding formed with multiple winding turn segments wound about the low friction inner sheath in a first direction about the longitudinal axis, the multilayer coil including a second coil layer having a second winding formed with multiple winding turn segments wound about the first winding in an opposite second direction about the longitudinal axis.

2. The lead of claim 1, wherein the second winding represents an outer winding wound concentrically about an exterior surface of the first winding that represents an inner winding.

3. The lead of claim 1, wherein at least the first winding includes a plurality of wires that are electrically insulated from one another, the plurality of wires being joined to corresponding electrodes from the multiple electrodes.

4. The lead of claim 1, wherein the first and second windings include a plurality of wires that are electrically insulated from one another and define a corresponding plurality of channels for at least one of sensing and delivering therapy.

5. The lead of claim 1, wherein the first and second windings shift, along the longitudinal axis, between elongated and condensed states as a distance between the distal and proximal ends changes with patient movement.

6. The lead of claim 1, wherein the first and second windings have a first length when in a condensed state and a second length when in an elongated state, the second length being approximately 10% to 50% greater than the first length.

7. The lead of claim 1, further comprising a biocompatible outer layer provided about an exterior surface of the second winding, the inner sheath and outer layer formed of polymers to stretch and contract when the first and second windings elongate and condense with patient movement.

8. The lead of claim 1, wherein the first direction is clockwise or counterclockwise about the longitudinal axis.

9. The lead of claim 1, wherein the multiple electrodes are arranged in an array, individual wires within the first and second windings being electrically coupled to the corresponding electrodes.

10. An implantable system, comprising:
an implantable medical device including pulse generating circuitry to generate electrical pulses as a neurostimulation therapy;
a lead body configured to be implanted in a patient, the lead body having a distal end and a proximal end, and a low friction inner sheath forming a lumen extending along a longitudinal axis between the distal and proximal ends;
a connector assembly provided at the proximal end of the lead body, the connector assembly configured to connect to the implantable medical device;
multiple electrodes provided proximate to the distal end of the lead body, the electrodes configured to at least one of deliver stimulating pulses and sense electrical activity; and
a multi-layer coil wound about the lumen and extending at least partially along a length of the lead body, the multi-layer coil including a first coil layer having a first winding formed with multiple winding turn segments wound about the low friction inner sheath in a first direction about the longitudinal axis, the multilayer coil including a second coli layer having second winding formed with multiple winding turn segments wound about the first winding in an opposite second direction about the longitudinal axis.

11. The system of claim 10, wherein the second winding represents an outer winding wound concentrically about an exterior surface of the first winding that represents an inner winding.

12. The system of claim 10, wherein at least the first winding includes a plurality of wires that are electrically insulated from one another, the plurality of wires being joined to corresponding electrodes from the multiple electrodes.

13. The system of claim 10, wherein the first and second windings include a plurality of wires that are electrically insulated from one another and define a corresponding plurality of channels for at least one of sensing and delivering therapy.

14. The system of claim 10, wherein the first and second windings shift, along the longitudinal axis, between elongated and condensed states as a distance between the distal and proximal ends changes with patient movement.

15. The system of claim 10, wherein the first and second windings have a first length when in a condensed state and a second length when in an elongated state, the second length being approximately 10% to 50% greater than the first length.

16. The system of claim 10, further comprising a biocompatible outer layer provided about an exterior surface of the second winding, the inner sheath and outer layer formed of polymers to stretch and contract when the first and second windings elongate and condense with patient movement.

17. The system of claim 10, wherein the first direction is clockwise or counterclockwise about the longitudinal axis.

18. The system of claim 10, wherein the multiple electrodes are arranged in an array, individual wires within the first and second windings being electrically coupled to the corresponding electrodes.

19. An implantable system, comprising:
an implantable medical device including pulse generating circuitry to generate electrical pulses as a neurostimulation therapy;
a lead body configured to be implanted in a patient, the lead body having a distal end and a proximal end, and a low friction inner sheath forming a lumen extending along a longitudinal axis between the distal and proximal ends;
a connector assembly provided at the proximal end of the lead body, the connector assembly configured to connect to the implantable medical device;
multiple electrodes provided proximate to the distal end of the lead body, the electrodes configured to at least one of deliver stimulating pulses and sense electrical activity; and
a multi-layer coil wound about the lumen and extending at least partially along a length of the lead body, the multi-layer coil including a first coil layer having a first plurality of conductors wound about the low friction inner sheath in a first direction about the longitudinal axis, the multilayer coil including a second coil layer having a second plurality of conductors wound about the first coil layer in an opposite second direction about the longitudinal axis, wherein the first and second plurality of conductors within the first and second coil layers translate along the longitudinal axis, between elongated and condensed states as a distance between the distal and proximal ends of the lead body changes with patient movement.
